(19) United States
(12) Patent Application Publication
(10) Pub. No.: US 2023/0402126 A1
(43) Pub. Date: Dec. 14, 2023

(54) MULTIPLE INSTANCE LEARNING FOR PEPTIDE-MHC PRESENTATION PREDICTION

(71) Applicant: NEC Laboratories Europe GmbH, Heidelberg (DE)

(72) Inventors: Jun Cheng, Heidelberg (DE); Brandon Malone, Heidelberg (DE)

(21) Appl. No.: 18/248,529

(22) PCT Filed: Mar. 12, 2021
(86) PCT No.: PCT/EP2021/056387

§ 371 Date: Apr. 11, 2023

(30) Foreign Application Priority Data
Oct. 13, 2020 (EP) 20201557.4

(51) Int. Cl. G16B 15/30; G16B 40/00

(52) U.S. Cl. CPC G16B 15/30; G16B 40/00

(57) ABSTRACT

A computer-implemented method for predicting binding and presentation of peptides by MHC molecules includes collecting training data, wherein the training data includes a set of MHC molecules in a sample as well as a set of observed peptide sequences that are presented by the MHC molecules, wherein it is unknown to which specific MHC molecules a peptide sequence is bound, and wherein the training data is organized in bags with each bag having a set of training instances. Labels are known for the bags, but unknown for the training instances. The method also uses a loss function to train a classifier at an instance-level, and predicts the label of new instances by applying the classifier directly and/or predicts the label of new bags by applying the MIL classifier to each instance of a respective bag and aggregates the results among all instances of the respective bag.

### CROSS REFERENCE TO RELATED APPLICATIONS

This application is a U.S. National Phase application under 35 U.S.C. § 371 of International Application No. PCT/EP2021/056387, filed on Mar. 12, 2021, and claims benefit to European Patent Application No. EP 20201557.4, filed on Oct. 13, 2020. The International Application was published in English on Apr. 21, 2022 as WO 2022/078633 A1 under PCT Article 21(2).

### FIELD

The present invention relates to a computer-implemented method and system for predicting binding and presentation of peptides by MHC molecules.

Furthermore, the present invention relates to a computer-implemented method for performing multiple instance learning, MIL.

### BACKGROUND

The adaptive immune system plays a central role in immune response against foreign molecules, such as pathogens or cancerous cells. The adaptive immune system has two major branches: humoral immunity, which concerns antibody generation, and cell-mediated immunity, which entails stimulation of cytotoxic CD8+ T cells among other things.

The major histocompatibility complex (MHC) class II plays an important role in both humoral and cell-mediated immunity (for reference, see Murphy, K. and Weaver, C., 2016. Janeway's immunobiology. Garland science). The primary role of MHC class II is to bind to and then present peptide sequences, which are short amino acid sequences, from exogenous proteins on the cell surface. This peptide-MHC complex leads to the stimulation of CD4+ T cells, or “helper T cells”. The helper T cells may then stimulate either the humoral or cell-mediated immune response pathways.

MHC class II molecules are mostly found in “professional” antigen presenting cells, such as dendritic cells. Among the MHC class II molecules, each person typically has two alleles each from the HLA-DQ and HLA-DP gene families, while they may have up to 10 alleles from the HLA-DR gene family (for reference, see Choo, S. Y., 2007. The HLA system: genetics, immunology, clinical testing, and clinical implications. Yonsei medical journal, 48(1), pp. 11-23). Importantly, different people have different MHC alleles, although some alleles are more common than others. The different versions of the MHC alleles have different amino acid sequences and structures, and these differences affect to which peptides the MHC alleles bind and present on the cell surface.

The presentation of peptides to T cells involves a series of processes. Important steps include binding between MHC molecules and peptides, as well as presentation of the peptide-MHC complex to the cell surface. Mass spectrometry can be used to detect peptides eluted from the cell surface to determine peptide presentation (for reference, see Purcell, A. W., Ramarathinam, S. H. and Ternette, N., 2019. Mass spectrometry-based identification of MHC-bound peptides for immunopeptidomics. Nature protocols, 14(6), p. 1687). Thousands of data points have been generated by such assays for hundreds of different MHC molecules (for reference, see Vita, R., Mahajan, S., Overton, J. A., Dhanda, S. K., Martini, S., Cantrell, J. R., Wheeler, D. K., Sette, A. and Peters, B., 2019. The immune epitope database (IEDB): 2018 update. Nucleic acids research, 47(D1), pp. D339-D343). As mentioned, each person has multiple MHC class II molecules; thus, typical mass spectrometry experiments cannot precisely identify the MHC molecule which presented a particular peptide. Another limitation of mass spectrometry is that it can only indicate peptides which were detected; that is, it cannot generate “negative” data points. It is therefore an important challenge to use this experimental data in order to train machine learning models to predict peptide-MHC presentation.

### SUMMARY

In an embodiment, the present disclosure provides a computer-implemented method for predicting binding and presentation of peptides by MHC molecules. The method comprises: collecting or generating training data, wherein the training data include a set of MHC molecules present in a biological sample as well as a set of observed peptide sequences that are presented by at least one of the MHC molecules present in the biological sample, wherein it is not known to which specific of the MHC molecules a peptide sequence is bound, and wherein the training data is organized in bags with each bag having a set of training instances, wherein labels are known for the bags, but unknown for the training instances; using a loss function to train an MIL classifier f_{θ} at an instance-level; and predicting the label of new instances by applying the MIL classifier f_{θ} directly and/or predicting the label of new bags by applying the MIL classifier f_{θ} to each instance of a respective bag and aggregating the results among all instances of the respective bag.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a schematic view illustrating a prediction scheme based on experimentally obtained data in accordance with an embodiment of the invention;

FIG. 2 is a schematic view illustrating bag label predictions by using a classifier predicting instance labels and by applying a pooling operation in accordance with an embodiment of the invention;

FIG. 3 is a schematic view illustrating a probability calibration function used to calibrate model confidence in accordance with an embodiment of the invention;

FIG. 4 is a schematic view illustrating a loss function modified to approximate negative samples with negative sampling in accordance with an embodiment of the invention; and

FIG. 5 is a schematic view illustrating a personalized cancer vaccine design in accordance with an embodiment of the invention.

### DETAILED DESCRIPTION

In accordance with an embodiment, the present invention improves and further develops methods and systems of the initially described type in such a way that the prediction performance is improved.

In accordance with another embodiment, the present invention provides a computer-implemented method for predicting binding and presentation of peptides by MHC molecules, the method comprising: collecting or generating training data, wherein the training data includes a set of MHC molecules present in a biological sample as well as a set of observed peptide sequences that are presented by at least one of the MHC molecules present in the biological sample, wherein it is not known to which specific of the MHC molecules a peptide sequence is bound, and wherein the training data are organized in bags with each bag having a set of training instances, wherein labels are known for the bags, but unknown for the training instances; using a loss function to train an MIL classifier f_{θ} at an instance-level; and predicting the label of new instances by applying the MIL classifier f_{θ} directly and/or predicting the label of new bags by applying the MIL classifier f_{θ} to each instance of a respective bag and aggregating the results among all instances of the respective bag.

Furthermore, in accordance with another embodiment, the present invention provides a computer-implemented method for performing multiple instance learning, MIL, the method comprising: collecting or generating training data, wherein the training data include bags with each bag having a set of training instances, wherein labels are known for the bags, but unknown for the training instances; training an MIL classifier at an instance-level by using a loss function that explicitly accounts for a model confidence in the model predictions during training, wherein individual training instances from positively labeled bags are weighted by a calibrated current model confidence function; and predicting the label of new instances by applying the MIL classifier directly and/or predicting the label of new bags by applying the MIL classifier to each instance of a respective bag and aggregating the results among all instances of the respective bag.

In further embodiments, a system for predicting binding and presentation of peptides by MHC molecules comprises one or more processors which, alone or in combination, are configured to allow for execution of any of the methods according to embodiments of the present invention.

In even further embodiments, a tangible, non-transitory computer-readable medium comprises instructions which, upon execution on one or more processors cause the one or more processors, alone or in combination, to allow for execution of any of the methods according to embodiments of the present invention.

Embodiments of the invention provide an MIL algorithm, with application to peptide—MHC predictions with multiple MHC alleles. Embodiments of the invention allow efficient usage of typical peptide—MHC mass spectrometry data with multiple potential allele labels. However, although the present disclosure focuses on predicting precisely binding and presentation of peptides by MHC alleles, which is an important step towards personalized T-cell-based vaccine design and immunotherapy, embodiments of the invention also relate to applications of an MIL algorithm in different contexts.

In an embodiment, the present invention provides a computer-implemented method for performing multiple instance learning, the method comprising a first step of collecting or generating training data where the labels are only known for bags of instances. The method may further include training a classifier at an instance-level where individual training instances from the positively labeled bags are weighted by a calibrated current model confidence in the loss function with the training data from the first step. Based on the trained MIL classifier the method may then include predicting the label of new instances by applying the instance-level classifier directly, or predicting the label of new bags by applying the instance-level classifier to each instance and aggregating the scores among all instances within the bags.

In an embodiment, the MIL classifier may be trained by using a loss function that explicitly accounts for model confidence in the model predictions during training. In the same or other embodiments, it may be provided that the probabilities are calibrated by means of a probability calibration function to accurately reflect the current model confidence. In this context it may be provided that training instances in the positively labeled bags are weighted by a calibrated current model confidence level.

There are several ways how to design and further develop the teaching of the present invention in an advantageous way. To this end it is to be referred to the dependent claims on the one hand and to the following explanation of preferred embodiments of the invention by way of example, illustrated by the figure on the other hand. In connection with the explanation of the preferred embodiments of the invention by the aid of the figure, generally preferred embodiments and further developments of the teaching will be explained.

Predicting the binding and presentation between MHC molecules and peptides is an important step towards T-cell-based vaccine design and immunotherapy. Given the importance of the problem and the availability of the data, many methods have been developed to predict MHC-peptide binding and peptide presentation. In some approaches, a single model is trained specifically for each MHC allele; other approaches instead train a single model covering all MHC alleles (pan model). The prediction performances of MHC class I models have reached a high level (auROC>0.98, for reference see Peters, M. E., Neumann, M., Iyyer, M., Gardner, M., Clark, C., Lee, K., and Zettlemoyer, L. (2018). Deep contextualized word representations. Proceedings of the 16th Annual Conference of the North American Chapter of the Association for Computational Linguistics: Human Language Technologies, 1, 2227-2237). On the other hand, models for class II still have limited performance. Despite recent progresses, there is still a need for better performing models. One significant limiting factor for MHC class II models is the limited amount of training data compared to class I. Thus, models that can efficiently use the limited available data and by transferring knowledge from other sources are extremely valuable.

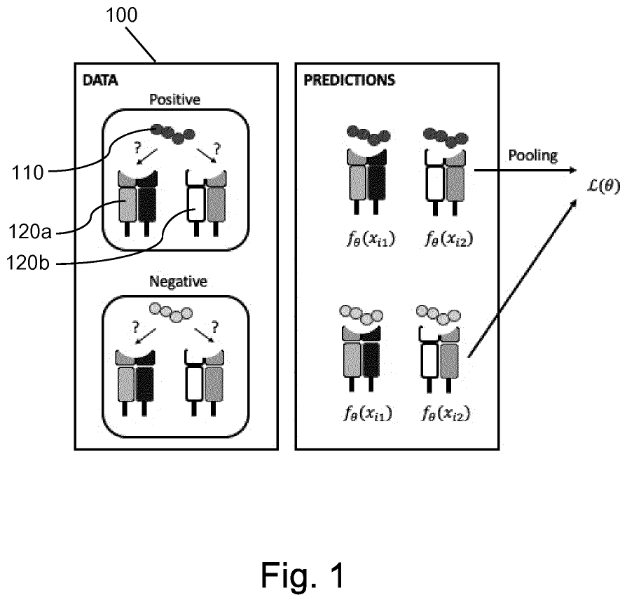

As already mentioned, predicting which peptide can or cannot be presented by which MHC molecule is crucial for neoantigen discovery and T-cell-based vaccines design, among other health-related problems. One important source of training data for such models is mass spectrometry. This technique identifies short peptides which are presented to the cell surface, due the MHC molecule(s) available in the cells. As indicated in the left part of FIG. 1, many mass spectrometry data 100 are generated with more than one MHC molecule in the cell, which means for a positive peptide 110 discovered with mass spectrometry, one or more MHC molecules 120a, 120b could be responsible for the presenting the peptide 110.

Embodiments of the present invention provide a method and a system which prioritize peptides for inclusion in a vaccine based on their likelihood to be presented on the cell surface by MHC molecules for a particular individual. In an embodiment, the prioritization is posed as a prediction problem, and a multiple instance learning (MIL) formulation is adopted to solve it. While prior work has also formulated this as an MIL problem, embodiments of the invention explicitly account for and calibrate model confidence during the learning process using a novel learning algorithm.

In standard supervised learning, labels are provided for each input sample. In some contexts, though, labels are instead assigned to sets or bags of inputs. In this setting, a bag of inputs is labeled as positive if it contains at least one positive input, otherwise the bag is labeled as negative.

As such, in accordance with an embodiment of the invention, the training data for multiple instance learning, MIL, may be defined as X={x₁, x₂, . . . , x_{N}} and the associated bag labels as {y₁, y₂, . . . , y_{N}}. Each bag may have a set of instances, i.e., Xᵢ={xᵢ₁, xᵢ₂, . . . , xᵢₘ}. MIL assumes each instance in the bag has a label yᵢⱼϵ{0,1}, but remains unknown in the training. Only labels yᵢ for the bag are provided, namely as follows:

$${y}_{i} = \{ \begin{matrix}1 & if ∃ j s . t . {y}_{ij} = 1 \\ 0 & Otherwise\end{matrix}$$

An MIL classifier f_{θ} can either learn to predict the label of a new bag f_{θ}(X) (bag-level approach) or to predict the label of an instance f_{θ}(xᵢⱼ) (instance-level approach). Embodiments of the invention focus on training classifiers predicting the label of instances, i.e. on the instance-level approach.

A classifier predicting the label of instances can be used to predict the label of a bag by applying a pooling operation h(⋅) on the predictions for all instances in the bag:

f_{θ}(X)=h(f_{θ}(xᵢ₁),f_{θ}(xᵢ₂), . . . ,f_{θ}(xᵢₘ)),

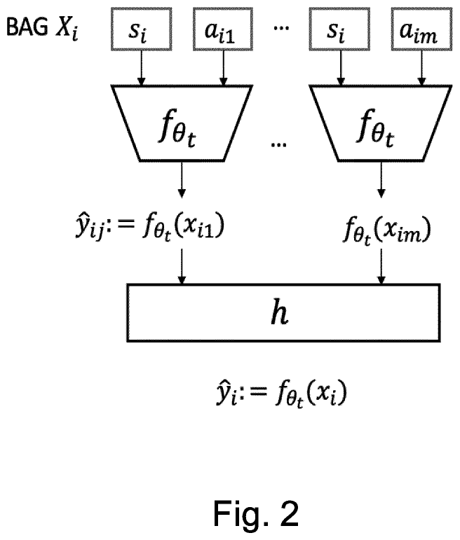

as indicated in the right part of FIG. 1 as well as in FIG. 2, where sᵢ denote peptide sequences, Aᵢ={a₁, . . . , aₘ} is a set of m MHC molecules associated with a biological sample, and yᵢ is a binary label indicating whether sᵢ was found to be presented by any of the MHC molecules in Aᵢ.

From the definition of the problem, h(⋅) is a permutation-invariant function, which means input order to the function has no influence on the result. The classifier f_{θ} may be trained using a loss function with the following form:

$$L ( θ ) = \frac{1}{N} {\sum }_{i = 1}^{N} {Loss}_{i} ( {y}_{i} , h ( {f}_{θ} ( {x}_{i 1} ) , {f}_{θ} ( {x}_{i 2} ) , … , {f}_{θ} ( {x}_{i m} ) ) ) ,$$

where N is the number of bags and M is the number of instances in each bag. Here, it should be noted that, in general, it is not required that all bags have the same number of instances.

According to some embodiments, the present invention provides methods and systems that include a multiple instance learning (MIL) approach based on the peptide-MHC presentation problem discussed above. The method may be performed in two phases, an offline training phase and an online prediction phase. In the offline training phase, a prediction model will be trained which explicitly accounts for and calibrates model confidence, which is in contrast to prior work. The trained model is then used during the online prediction phase.

According to some embodiments, the present invention provides methods and systems for predicting binding and presentation of peptides by MHC molecules that are configured to receive, as input in the offline training phase, a set of observed peptides which are presented by at least one MHC molecule which was present in a biological sample, as well as the set of MHC molecules which are present in that sample. As already explained above, it is not known, however, to which specific MHC molecule a peptide was bound. This is exactly the kind of data produced by mass spectrometry experiments.

In an embodiment, a standard approach may be used to generate negative examples for training. It should be noted, however, that the applicability of the approach proposed in accordance with the present invention does not depend on how negative examples are created.

More specifically, the input may be provided in the form of a set of triples {sᵢ, Aᵢ, yᵢ}, where sᵢ is a peptide sequence, Aᵢ={a₁, . . . , aₘ} is a set of m MHC molecules associated with a biological sample, and yᵢ is a binary label indicating whether sᵢ was found to be presented by any of the MHC molecules in Aᵢ.

The goal of the offline training phase is to train a machine learning model f_{θ} which takes as input Xᵢ=(sᵢ, Aᵢ) and correctly predicts yᵢ. One example of f_{θ} is a pretrained bidirectional encoder representations from transformers (BERT) model. However, as will be appreciated by those skilled in the art, other model types are likewise possible. The only restriction is that the model provide a probability p(yᵢⱼ=1|sᵢ, aⱼ) associated with the prediction for each instance.

According to an embodiment of the invention, each peptide is associated with a bag of alleles. The bag is labeled as positive if at least one of the allele presented the peptide, otherwise the bag is labelled as negative. The training data may be modelled as a multiple instance learning (MIL) problem. Here, the ith bag with m alleles is denoted as Aᵢ={aᵢ₁, aᵢ₂, . . . , aᵢₘ} and the corresponding peptide sequence as sᵢ. At each training step, the probability p(yᵢⱼ=1|xᵢⱼ) of every instance (aᵢⱼ, sᵢ) in the bag may be predicted as ŷᵢⱼ=f_{θ}(aᵢⱼ, sᵢ) with the neural network model f_{θ}. A symmetric pooling operator may be used to pool the prediction of the bag from the predictions of instances within it. To incorporate the uncertainty of the deconvolution operation, at each training epoch each positive data point i from deconvolution may be weighted by a calibrated predicted probability of being positive [symbol] (\hat{p}ᵢ).

According to an embodiment of the invention, the parameters of the model may then be learned according to the following loss function:

$$ℒ ( θ ) = - \frac{1}{{N}_{P o s}} {\sum }_{i ∈ P o s}^{{N}_{P o s}} ( 𝒞 ( {\hat{p}}_{i} ) \cdot w \cdot log ( {\hat{y}}_{i} ) ) - \frac{1}{{N}_{N e g}} {\sum }_{i ∈ N e g}^{{N}_{N e g}} \frac{1}{{m}_{i}} {\sum }_{j = 1}^{{m}_{i}} log ( 1 - {\hat{y}}_{ij} ) ,$$

where

$${\hat{y}}_{i} = {max}_{j} ( {f}_{θ} ( {x}_{ij} ) ) ,$$

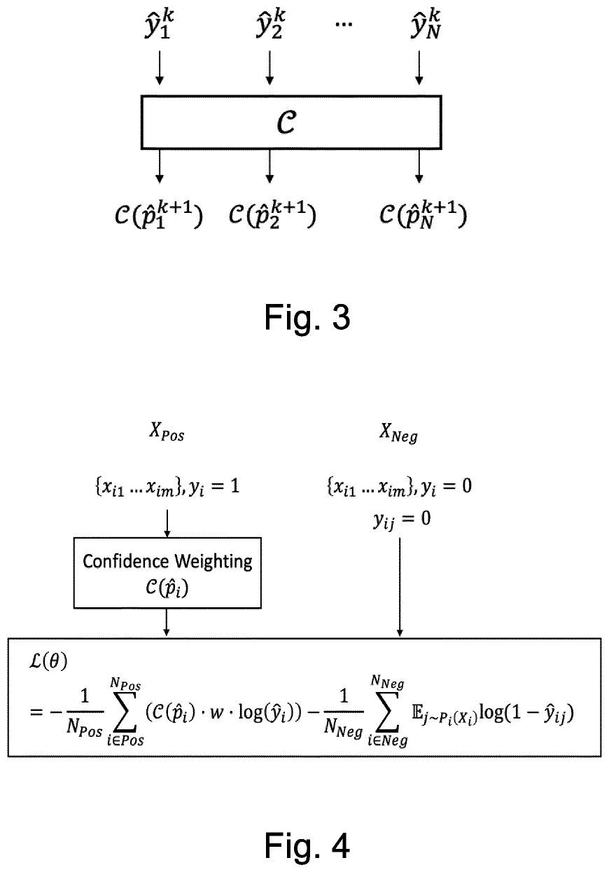

\hat{p}ᵢ is the predicted probability of ŷᵢ of the previous training epoch of the model, [symbol] is a probability calibration function (FIG. 3), w is the weight for the positive class to count for class imbalance, and xᵢⱼ corresponds to the tuple (sᵢ, aᵢ), where sᵢ is the peptide and aᵢ is the jₜₕ MHC molecule in Aᵢ. According to the embodiment illustrated in FIG. 3, the probability calibration function [symbol] may be configured to receive as input the values ŷᵢ of a current training epoch k of the model and may calculate calibrated probabilities \hat{p}ᵢ for a subsequent training epoch k+1 of the model. With respect to the instance weighting it should be noted that only the instances in positively labeled bags are weighted with calibrated model confidences, in accordance with embodiments of the invention, while negative samples are not weighted (since there is no uncertainty with the labels of negative classes). In this context it may be provided that either all negative samples are used or that negative sampling is performed if negative bags are large and computation is limited.

The given formulation incorporates all negative instances in all of the negative bags. However, in cases for which there are many negative bags, this is computationally challenging. Therefore, according to an alternative embodiment, it may be provided to approximate the negative samples with negative sampling, as shown in FIG. 4. Accordingly, the above loss function may be modified as:

$$ℒ ( θ ) = - \frac{1}{{N}_{P o s}} {{\sum }_{i ∈ Po s}}^{{N}_{P o s}} ( 𝒞 ( {\hat{p}}_{i} ) \cdot w \cdot log ( {\hat{y}}_{i} ) ) - \frac{1}{{N}_{N e g}} {{\sum }_{i ∈ Ne g}}^{{N}_{N e g}} {𝔼}_{j \sim {P}_{i} ( {X}_{i} )} log ( 1 - {\hat{y}}_{ij} )$$

For computational reason, negative sampling may be performed with a probability distribution Pᵢ(Xᵢ) instead of using all negative samples. According to an embodiment, for the MHC-peptide presentation problem, one may choose to use the following delta distribution for Pᵢ(Xᵢ)=Pᵢ(xᵢ₁, xᵢ₂, . . . , xᵢₘ):

$${P}_{i} ( {x}_{i j} ) = \{ \begin{matrix}1 & if {f}_{θ} ( {x}_{ij} ) = max ( {f}_{θ} ( {x}_{i 1} ) , {f}_{θ} ( {x}_{i 2} ) , … , {f}_{θ} ( {x}_{i m} ) ) \\ 0 & otherwise\end{matrix}$$

That is, the method uses the most likely positive example predicted by the current model from the negative bag.

Considering the above, a multiple instance learning (MIL) algorithm according to an embodiment of the invention, with application to peptide MHC predictions with multiple MHC alleles, can be stated as follows:

Algorithm: Probability Reweighted Multiple Instance Learning

```
Input: Training data {Xᵢ, yᵢ}_{i∈1} _{. . . N}, where Xᵢ := {sᵢ, Aᵢ}, yᵢ ∈ {0, 1};
Random initalize θ₀ or transfer θ₀ from a related task, θₖ ← θ₀, choose w
while not converge do:
  for k in 0 . . . N_{EPOCH}:
    Predict bag labels with the current model \hat{P} := {h(f_{θ}_{ₖ}(xᵢⱼ) . . . , f_{θ}_{ₖ}(xᵢₘ))}_{i∈1 . . . N}
    Train a probability calibration model [symbol]    ₖ with {yᵢ, logit(\hat{p}ᵢ)}_{i∈1 . . . N} as input
    θₜ ← θₖ
    for t in 0 . . . N_{BATCH}:
      \hat{p}ᵢ := \hat{P}[i], ŷᵢⱼ := f_{θ}_{ₜ}(xᵢⱼ), yᵢ := h({yᵢⱼ}_{j∈1 . . . m})
      $ℒ ( {θ}_{t} ) = - \frac{1}{{N}_{P o s}} {\sum }_{i ∈ P o s}^{{N}_{P o s}} ( {𝒞}_{k} ( {\hat{p}}_{i} ) \cdot w \cdot log ( {\hat{y}}_{i} ) ) - \frac{1}{{N}_{N e g}} {\sum }_{i ∈ N e g}^{{N}_{N e g}} {𝔼}_{j \sim {P}_{i} ( {X}_{i} )} log ( 1 - {\hat{y}}_{i j} )$
      θₜ ← ∇_{θ}_{ₜ} [symbol]   (θₜ)
    end for
  θₖ ← θₜ
end for
return θ
```

It is important to note that, compared to prior art, the loss function L(θ) according to the invention explicitly accounts for the model confidence in the model predictions during training. In accordance with embodiments of the invention this is achieved by accounting for \hat{p}ᵢ, the predicted probability of ŷᵢ. Specifically, in existing approaches, the loss can be attributed to wrong instances xᵢⱼ, therefore f_{θ} can be optimized to predict a “correct” label of the bag by predicting on the wrong instance xᵢⱼ.

Further, embodiments of the invention also extend prior art by including the function [symbol] for calibrating the predicted probabilities. The probabilities \hat{p}ᵢ can be calibrated by performing isotonic regression from the predicted logits (i.e. the logarithms of the odds (\hat{p}ᵢ/(1−\hat{p}ᵢ))) and the labels on the training set. For instance, the isotonic regression may be performed according to the approach described in Barlow, R. E., 1972. Statistical inference under order restrictions; the theory and application of isotonic regression (No. 04; QA278. 7, B3.), the entire contents of which is hereby incorporated by reference herein. However, as will be appreciated by those skilled in the art, other approaches such as Platt's scaling could also be used. The applicability of the approach proposed in accordance with the present invention does not depend on the exact form of the calibration function.

The parameters θ of the model can then be learned using appropriate optimization techniques to minimize this loss function. For example, if f_{θ} is differentiable, such as with the BERT model, then gradient descent or similar algorithms can be used. According to an alternative embodiment, if f_{θ} is not differentiable, then Bayesian optimization or other black box methods can be used. The applicability of the approach proposed in accordance with the present invention does not depend on whether f_{θ} is differentiable.

After termination of the offline training phase as described above, an online prediction phase can be conducted. Specifically, after training, the model f_{θ} takes as input Xᵢ and predicts the label yᵢ. That is, the model takes as input a peptide sequence and a set of MHC molecules, and it predicts whether that peptide will be presented by any of those MHC molecules. According to embodiments it may be provided that the MIL classifier f_{θ} is used to make predictions for all combinations of peptide sequences and MHC molecules present in a biological sample. Based on thereupon, the peptides with the highest likelihood of being presented may be determined as candidates for being synthesized and included in a personalized cancer vaccine.

In practice, presentation of a peptide by an MHC molecule is only one (very important) step among many in ultimately creating an effective cancer vaccine. Predictive models for many of those steps do not obviously entail a multiple instance learning problem. Thus, the approach proposed in accordance with the embodiment of the present invention may be only applicable for parts of the vaccine design process. Furthermore, it should be noted that the proposed approach includes models which output some notion of probability. While this is common for classification problems, it is much less common for regression problems. Thus, the approach may be of limited use for multiple instance regression learning problems. Still further, it should be noted that most probability calibration functions may require access to all uncalibrated probabilities. Thus, minibatch optimization approaches, which update the model after making predictions on only a few training samples, may not be compatible with certain embodiments of the present invention approach. Instead, embodiments of the invention train a calibration model at the beginning of each epoch.

The current state of the art for multiple instance learning for peptide—MHC presentation is the work by Reynisson, B., Alvarez, B., Paul, S., Peters, B. and Nielsen, M., 2020. NetMHCpan-4.1 and NetMHCIIpan-4.0: improved predictions of MHC antigen presentation by concurrent motif deconvolution and integration of MS MI-C eluted ligand data. Nucleic Acids Research, the entire contents of which is hereby incorporated by reference herein. However, their approach does not incorporate the confidence weighting or calibration operations. Empirically, it could be demonstrated that the approach according to the present invention outperforms the approach by Reynisson et al on a variety of datasets.

### MHC Class II Binding Data

In accordance with embodiments of the invention, to train the MHC class II binding model, the data from Jensen et al., 2018 (see Jensen, K. K., Andreatta, M., Marcatili, P., Buus, S., Greenbaum, J. A., Yan, Z., Sette, A., Peters, B., and Nielsen, M. (2018). Improved methods for predicting peptide binding affinity to MHC class II molecules. Immunology, 154(3), 394-406, the entire contents of which is hereby incorporated by reference herein) were used, since it has been designed to minimize the overlap between the training and evaluation sets. The original data was collected from the Immune Epitope Database (IEDB, Vita, R., Mahajan, S., Overton, J. A., Dhanda, S. K., Martini, S., Cantrell, J. R., Wheeler, D. K., Sette, A., and Peters, B. (2019). The Immune Epitope Database (IEDB): 2018 update. Nucleic Acids Research, 47(D1), D339-D343, accessed on 30 Jun. 2020) up to the year 2016. The data consists of 134 281 data points and covers HLA-DR, HLA-DQ, HLA-DP and H-2 mouse MHC allele. The affinity labels were transformed from IC50 to value between 0 and 1 with the formula 1−log(IC50)/log(50 000).

The data from Jensen et al. was collected from IEDB up to the year 2016. To benchmark on an independent dataset where no model has been used for training or validation, quantitative binding data were collected from IEDB and data already used in Jensen et al. were filtered out. In addition, additional independent binding data from the Dana-Farber repository (for reference, see G. L., Lin, H. H., Keskin, D. B., Reinherz, E. L., and Brusic, V. (2011). Dana-farber repository for machine learning in immunology. Journal of immunological methods, 374(1-2), 18-25, the entire contents of which is hereby incorporated by reference herein) were collected. In the end, 2 413 additional MHC-peptide pairs covering 47 MHC class II alleles were collected.

### MHC Class II Presentation Data

To train a MHC class II mass spectrometry presentation model, the data curated from Reynisson, B., Alvarez, B., Paul, S., Peters, B., and Nielsen, M. (2020). NetMHCpan-4.1 and NetMHCIIpan-4.0: improved predictions of MHC antigen presentation by concurrent motif deconvolution and integration of MS MHC eluted ligand data. Nucleic Acids Research, pages 1-6., the entire contents of which is hereby incorporated by reference herein, were used. The original data were curated from IEDB and other public sources. The data covers 41 MHC class II allele with peptide length ranging from 13 to 21. Each data point consists of the peptide ligand, the source protein and list of possible MHC class II allele bound to the peptide. The data points where only one MHC allele is unambiguously given are referred as single-allele data (SA), whereas the data points where multiple potential alleles, due to the nature of the mass spectrometry experiment, are given are referred as multi-allele data (MA). Reynisson et al., selected negative peptides by randomly sampling from the UniProt database. Peptide lengths for the negatives were sampled uniformly from 13 to 21.

According to embodiments of the invention, the MIL problem is tackled with an instance-level approach. Compared to a bag-level approach, this approach maximizes the model accuracy at predicting one single instance instead of a whole bag. Performance of an instance-level approach relies on correctly detecting the key instance (the positive instance in the positive bag). Therefore, a good instance-level model can not only be applied to the MIL problem but also to the single instance learning problem. In fact, in the peptide—MHC presentation problem, embodiments of the invention provide for using the same model jointly trained on single instance data and multiple instance data to maximize the usage of existing data. Previous work has shown that models which detect key instances also have better bag-level generalizability. Bag-level approaches, however, may have good performance at the bag-level, but are not guaranteed to generalize well to single instance cases. For biological applications, it is crucial for that the model is able to detect correctly the key instances.

In the following, some further example embodiments from several domains in which the invention can be used will be described.

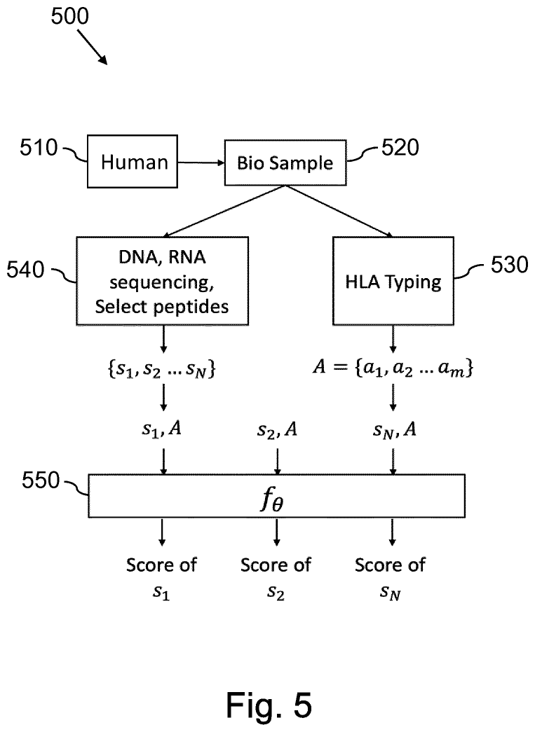

Personalized cancer vaccine design. This embodiment relates to a personalized cancer vaccine design system 500, which is schematically illustrated in FIG. 5, wherein the model is trained as described above. For prediction, the set of MHC molecules (generally denoted HLA, Human Leukocyte Antigen, Typing 530 in FIG. 5) is taken as the MHC molecules from a biological sample 520 taken from a specific patient 510, and the set of peptides 540 are based on mutations present in the cancerous cells of the patient 510. Predictions are made as described for all combinations of peptide and MHC pairs for that patient by using the trained MIL classifier f_{θ}, as shown at 550. The peptides with the highest likelihood of being presented (i.e. with the highest scores, as indicated in FIG. 5) are then synthesized and included in a personalized cancer vaccine for that specific patient 510.

Immune response prediction. ELISpot is a widely-used immune response assay which measures if a particular peptide leads to an immune response when combined with a biological sample, such as blood from a patient infected with coronavirus. For example, interferon gamma is commonly measured with ELISpot. The immune response measurement from ELISpot is a result of interactions between the peptide and at least one of the MHC molecules present in the sample. According to an embodiment, the MIL approach discloses herein can also be used to train a model to predict this immune response. Compared to the formulation above, the only difference is that the bag labels are the results of the immune response assays. Such a model could also be used in a personalized cancer vaccine design system.

Histopathology-based cancer diagnosis. Histopathology stains are created by taking slices of tissue from a biological sample, and then staining them using chemicals such as hematoxylin and eosin. The stained images can then be used to identify features such as the nuclei of cells and extracellular support structures like collagen. These stained images can also be used to train machine learning models to predict whether a particular tissue slice contains cancer or not, i.e., cancer diagnosis. However, the stained images are typically much too large for current hardware to process at once, and they are consequently split into “patches” for learning. Typically, not all patches from a single stained image will contain a cancerous region, even though other patches from that image do.

This can also be thought of as a multiple instance learning problem, in which a single stained image corresponds to each bag, and the patches are the individual instances within the bags. The label on the bag indicates whether cancer is present in that stained image. According to embodiments of the invention, such a predictive model may be used in a cancer diagnosis system.

Document classification. Document classification tasks take documents as input and classify documents into predefined categories. According to embodiments, MIL can be applied by considering paragraphs or sentences as instances and the documents as bags. Example labels could be the topic of the document, such as “politics”, “sports”, or “science”. It is noted here that this example demonstrates that the approach proposed in accordance with embodiments of the invention can be used to classify tasks with more than two classes, by making the obvious changes in the loss function. Further, this example demonstrates that the approach can be easily generalized to multi-label classification. For example, a document may be associated with both “politics” and “sports”. In this case, embodiments of the invention may simply treat each label as a binary classification, and the loss function may be replicated for each label.

In further embodiments, a system for predicting binding and presentation of peptides by MHC molecules or a system for performing multiple instance learning comprises one or more processors which, alone or in combination, are configured to allow for execution of any of the methods according to embodiments of the present invention. In even further embodiments, a tangible, non-transitory computer-readable medium comprises instructions which, upon execution on one or more processors cause the one or more processors, alone or in combination, to allow for execution of any of the methods according to embodiments of the present invention. The processors can include one or more distinct processors, each having one or more cores, and access to memory. Each of the distinct processors can have the same or different structure. The processors can include one or more central processing units (CPUs), one or more graphics processing units (GPUs), circuitry (e.g., application specific integrated circuits (ASICs)), digital signal processors (DSPs), and the like. The processors can be mounted to a common substrate or to multiple different substrates. Processors are configured to perform a certain function, method, or operation (e.g., are configured to provide for performance of a function, method, or operation) at least when one of the one or more of the distinct processors is capable of performing operations embodying the function, method, or operation. Processors can perform operations embodying the function, method, or operation by, for example, executing code (e.g., interpreting scripts) stored on memory and/or trafficking data through one or more ASICs. Processors can be configured to perform, automatically, any and all functions, methods, and operations disclosed herein. Therefore, processors can be configured to implement any of (e.g., all) the protocols, devices, mechanisms, systems, and methods described herein. For example, when the present disclosure states that a method or device performs task “X” (or that task “X” is performed), such a statement should be understood to disclose that processor is configured to perform task “X”.

Each of the computer entities can include memory. Memory can include volatile memory, non-volatile memory, and any other medium capable of storing data. Each of the volatile memory, non-volatile memory, and any other type of memory can include multiple different memory devices, located at multiple distinct locations and each having a different structure. Memory can include remotely hosted (e.g., cloud) storage. Examples of memory include a non-transitory computer-readable media such as RAM, ROM, flash memory, EEPROM, any kind of optical storage disk such as a DVD, magnetic storage, holographic storage, a HDD, a SSD, any medium that can be used to store program code in the form of instructions or data structures, and the like. Any and all of the methods, functions, and operations described in the present application can be fully embodied in the form of tangible and/or non-transitory machine-readable code (e.g., interpretable scripts) saved in memory.

Many modifications and other embodiments of the invention set forth herein will come to mind to the one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1: A computer-implemented method for predicting binding and presentation of peptides by major histocompatibility complex (MHC) molecules, the method comprising:
collecting or generating training data, wherein the training data includes a set of MHC molecules present in a biological sample as well as a set of observed peptide sequences that are presented by at least one of the MHC molecules present in the biological sample, wherein it is not known to which specific of the MHC molecules a peptide sequence is bound, and wherein the training data are organized in bags with each bag having a set of training instances, wherein labels are known for the bags, but unknown for the training instances;
using a loss function to train a multiple instance learning (MIL) classifier f_{θ} at an instance-level; and
predicting the label of new instances by applying the MIL classifier f_{θ} directly and/or predicting the label of new bags by applying the MIL classifier f_{θ} to each instance of a respective bag and aggregating the results among all instances of the respective bag.

2: The method according to claim 1, wherein the MIL classifier f_{θ} is trained by using the loss function (L) of the form:
$$L ( θ ) = \frac{1}{N} {\sum }_{i = 1}^{N} {Loss}_{i} ( {y}_{i} , h ( {f}_{θ} ( {x}_{i 1} ) , {f}_{θ} ( {x}_{i 2} ) , … , {f}_{θ} ( {x}_{i m} ) ) ) ,$$
wherein xᵢ₁, xᵢ₂, . . . , xᵢₘ are the instances of bag i, yᵢ are the associated bag labels, h is a permutation-invariant pooling function, N is the number of bags and M is the number of instances in each bag.

3: The method according to claim 1, wherein the loss function explicitly accounts for a model confidence in the model predictions during training.

4: The method according to claim 3, wherein individual training instances from positively labeled bags are weighted by a calibrated current model confidence function.

5: The method according to claim 1, wherein the training data is provided in form of a set of triples {sᵢ, Aᵢ, yᵢ}, where sᵢ is a peptide sequence, Aᵢ={a₁, . . . , aₘ} is a set of m MHC molecules associated with a biological sample, and yᵢ is a binary label indicating whether sᵢ was found to be presented by any of the MHC molecules in Aᵢ.

6: The method according to claim 1, further comprising obtaining the training data from mass spectrometry experiments.

7: The method according to claim 5, further comprising:
training the parameters of the MIL classifier f_{θ} by the loss function L(θ) that includes a probability calibration function C configured to predict in each training epoch k+1 the probabilities \hat{p}ᵢ of ŷᵢ of the previous training epoch k, wherein
$${\hat{y}}_{i} = {max}_{j} ( {f}_{θ} ( {x}_{ij} ) )$$
and xᵢⱼ corresponds to the tuple (sᵢ, aᵢ), where sᵢ is the peptide and aᵢ is the jₜₕ MHC molecule in Aᵢ.

8: The method according to claim 1, further comprising:
providing, in a prediction phase after training, the MIL classifier f_{θ} a peptide sequence sᵢ and a set of MHC molecules aᵢ as input, and
predicting, by applying the MIL classifier f_{θ} to the input, whether the peptide sequence sᵢ will be presented by any of the MHC molecules aᵢ.

9: The method according to claim 1, further comprising:
using the MIL classifier f_{θ} to make predictions for all combinations of peptide sequences and MHC molecules present in the biological sample; and
determining the peptides with the highest likelihood of being presented as candidates for being synthesized and included in a personalized cancer vaccine.

10: A tangible, non-transitory computer-readable medium storing processor-executable instructions which, when executed, allow for performance of the method according to claim 1.

11: A system for predicting binding and presentation of peptides by major histocompatibility complex (MHC) molecules, the system comprising one or more processors which, alone or in combination, are configured to allow for execution of a method comprising:
collecting or generating training data, wherein the training data includes a set of MHC molecules present in a biological sample as well as a set of observed peptide sequences that are presented by at least one of the MHC molecules present in the biological sample, wherein it is not known to which specific of the MHC molecules a peptide sequence is bound,
organizing the training data in bags, with each bag having a set of training instances, wherein labels are known for the bags, but unknown for the training instances;
using a loss function to train a multiple instance learning (MIL) classifier f_{θ} at an instance-level; and
predicting the label of new instances by applying the MIL classifier f_{θ} directly and/or predicting the label of new bags by applying the MIL classifier f_{θ} to each instance of a respective bag and aggregating the results among all instances of the respective bag.

12: A computer-implemented method for performing multiple instance learning, multiple instance learning (MIL), the method comprising:
collecting or generating training data, wherein the training data includes bags with each bag having a set of training instances, wherein labels are known for the bags, but unknown for the training instances;
training an MIL classifier at an instance-level by using a loss function that explicitly accounts for a model confidence in the model predictions during training, wherein individual training instances from positively labeled bags are weighted by a calibrated current model confidence function; and
predicting the label of new instances by applying the MIL classifier directly and/or predicting the label of new bags by applying the MIL classifier to each instance of a respective bag and aggregating the results among all instances of the respective bag.

13: The method according to claim 12, wherein the training data includes a set of major histocompatibility complex (MHC) molecules present in a biological sample as well as a set of observed peptide sequences that are presented by at least one of the MHC molecules present in the biological sample, wherein it is not known to which specific of the MHC molecules a peptide sequence is bound; and
wherein the MIL classifier is trained to predict whether a particular peptide sequence will be presented by any of the MHC molecules present in the biological sample.

14: The method according to claim 12, wherein the training data are generated by an immune response assay that measures whether a particular peptide leads to an immune response when combined with a biological sample; and
wherein the MIL classifier is trained to predict the immune response.

15: The method according to claim 12, wherein the training data include a set of stained images of histological samples obtained by taking slices of tissue from a biological sample, wherein the stained images are split into patches, and
wherein the MIL classifier is trained to predict whether or not a particular patch of a stained image contains a cancerous region; or
wherein the training data include a set of text documents, wherein each of the documents is considered to represent a bag of the training data and the paragraphs and/or sentences of the documents are considered to represent the training instances of the respective bag, and
wherein the MIL classifier is trained to predict a topic of the documents.
